(19) United States
(12) Patent Application Publication
(10) Pub. No.: US 2015/0366191 A1
(43) Pub. Date: Dec. 24, 2015

(54) ANTIMICROBIAL METHOD BY BLOCKING MANNITOL METABOLISM AND ANTIMICROBIAL COMPOSITION CONTAINING MANNITOL METABOLIC INHIBITOR

(71) Applicant: Research & Business FOUNDATION SUNGKYUNKWAN UNIVERSITY, Suwon-si (KR)

(72) Inventors: Kyeong Kyu KIM, Suwon-si (KR); Trung Thanh NGUYEN, Suwon-si (KR); Jongkeun CHOI, Suwon-si (KR)

(73) Assignee: Research & Business FOUNDATION SUNGKYUNKWAN UNIVERSITY, Suwon-si (KR)

(21) Appl. No.: 14/530,212

(22) Filed: Oct. 31, 2014

(30) Foreign Application Priority Data
Jun. 23, 2014 (KR) 10-2014-0076570

(51) Int. Cl. A01N 31/02; A61K 8/49; A61K 8/34; A01N 47/36; A61Q 17/00; A61K 8/42; A01N 47/30; A01N 45/00; A61K 8/63; A01N 43/90; A01N 43/42

(52) U.S. Cl. CPC A01N 31/02; A01N 43/90; A61K 8/498; A61K 8/345; A01N 47/36; A61K 8/4966; A61K 8/4926; A01N 43/42; A61K 8/42; A01N 47/30; A01N 45/00; A61K 8/63; A61Q 17/005; A61K 2800/592

(57) ABSTRACT

The present invention relates to an antimicrobial method of killing bacteria having a mannitol metabolic pathway by inhibition of a mannitol metabolism, a method of screening a mannitol metabolic inhibitor, and an antimicrobial composition and cosmetic material containing a mannitol metabolic inhibitor. More particularly, the present invention relates to an antimicrobial method targeted at mannitol dehydrogenase such as mannitol-1-phosphate-5-dehydrogenase (M1PDH), a method of screening an inhibitor, and a composition. Therefore, the antimicrobial method, antimicrobial composition, and cosmetic material which solve a problem of antibiotic resistance and have an excellent antimicrobial effect may be provided.

### BACKGROUND

1. Field of the Invention

The present invention relates to an antimicrobial method of killing bacteria having a mannitol metabolic pathway by inhibition of a mannitol metabolism, a method of screening a mannitol metabolic inhibitor, and an antimicrobial composition and cosmetic material containing a mannitol metabolic inhibitor. More particularly the present invention relates to an antimicrobial method targeted at mannitol dehydrogenase such as mannitol-1-phosphate-5-dehydrogenase (M1PDH), a method of screening an inhibitor, and a composition.

2. Discussion of Related Art

Antimicrobials are widely used to treat a disease caused by bacterial infection, and have been isolated from natural substances including fungi or chemically synthesized. The most representative antimicrobial is penicillin which was found by Alexander Fleming in 1928. The penicillin is an antimicrobial first developed by mankind, which has an antimicrobial effect of inhibiting growth of bacteria. Specifically, a part of β-lactam of the penicillin optionally binds to a transpeptidase linking peptidoglycan molecules, which become a cell membrane of bacteria, to prevent growth of the cell wall of the bacteria, resulting in a weak cell wall, which cannot stand osmotic pressure, and bacteriolysis. After the development of the penicillin, excellent antimicrobials are being developed based on this, and a representative one thereof is methicillin produced by modifying a part of the chemical structure of the penicillin. While antimicrobials serve to various roles to prevent or treat bacterial infection by mechanisms such as blocking of synthesis of a cell wall, breakdown of a structure of a cell membrane, inhibition of DNA or RNA synthesis, and inhibition of protein synthesis, due to resistance to a conventional antimicrobial caused by the indiscriminate use of antimicrobials, antimicrobial-resistant bacteria emerged. The antimicrobial-resistant bacteria have a resistance to a specific antimicrobial, and thus a suitable drug effect is not obtained. Since the antimicrobial-resistant bacteria transfer the resistance to other bacteria, the number of antimicrobial-resistant bacteria is increasing. Therefore, once bacteria have a resistance, a stronger antimicrobial or a different type of antimicrobial should be used.

Representative examples of various bacteria having a resistance to an antimicrobial include methicillin-resistant Staphylococcus aureus(MRSA), vancomycin-resistant enterococci (VRE), vancomycin-intermediate Staphylococcus aureus (VISA), and E. coli P157:H7 having a resistance to at least 6 types of antimicrobials.

MRSA was beginning to be reported in all over the world since first found in the United Kingdom in 1961, and can be treated by only a limited antimicrobial since it has a resistance to most of antimicrobials as well as methicillin. In addition, MRSA is the most frequently emerging causative organism among pathogenic bacteria triggering in-clinic infection, and may be critical to lead to death when infected to patients with weak immune systems or the old. For example, it was reported that, in the year of 2006, in the United States, approximately 94,000 persons were infected by MRSA, and approximately 19,000 persons or more among them died. In Korea, it was reported that MRSA is an infectious pathogenic organism emerging in a hospital, and detected in almost 80% of large general hospitals.

For this reason, development of new antimicrobial method and drug having a new action mechanism which does not exhibit a resistance to an antimicrobial becomes an important issue, and a variety of research is being made (Korean Patent Laid-Open Application No. 10-2006-0069790), but there are still a lot of problems left to figure out.

### SUMMARY OF THE INVENTION

The inventors identified that viability of Staphylococcus depleted of M1PDH, which is one of the critical enzymes involved in a mannitol metabolism, is considerably decreased in an environment with a high concentration of mannitol, and thus found that bacteria can be controlled by inhibition of the mannitol metabolism. Therefore, the inventors invented a method of screening a compound inhibiting the mannitol metabolism, and a method of effectively killing bacteria using a screened inhibitor. Particularly, substrate specificity was investigated using a three-dimensional structure of an M1PDH protein, and the compound inhibiting M1PDH was found by virtual screening using a three-dimensional structure and by screening of an inhibitor using a compound library, and its effect was identified. Thus, the present invention was completed.

Accordingly, the present invention is directed to providing an antimicrobial method of killing bacteria through inhibition of a mannitol metabolism and an antimicrobial pharmaceutical/cosmetic composition solving an antibiotic resistance problem and having an excellent antimicrobial effect as an antimicrobial composition, unlike conventional antibiotics.

Specifically, the present invention is directed to providing an antimicrobial method by the inhibition of a mannitol metabolism by treating bacteria cultured out of cells with 55 to 500 mM of mannitol, or treating bacteria cultured with a microphage with 2.74 to 164 mM of mannitol as well as a mannitol dehydrogenase inhibitor.

However, technical objects to be achieved by the present invention are not limited to the above-described objects, and other objects not described herein will be clearly understood by those of ordinary skill in the art from descriptions below.

In one aspect, the present invention provides a method of killing bacteria having a mannitol metabolic pathway by inhibiting a mannitol metabolism.

In one embodiment of the present invention, the method includes treating the bacteria with a mannitol metabolic inhibitor and 55 to 500 mM of mannitol.

In another embodiment of the present invention, the treatment is performed in vitro.

In still another embodiment of the present invention, the bacteria having a mannitol metabolic pathway are selected from the group consisting of Staphylococcus aureus, Staphylococcus haemolyticus, Staphylococcus saprophyticus, Streptococcus mutants, Streptococcus pneumoniae, Streptococcus pyogenes, Bacillus anthracis, Pseudomonas aeruginosa, Pseudomonas stutzeri, Vibrio cholera, Vibrio vulnificus, Vibrio parahaemolyticus, Shigella flexneri, Yersinia enterocolitica, Yersinia pesti, Aeromonas salmonicida, Mycoplasma mycoides, Enterococcus faecalis, Yersinia pseudotuberculosis, Pasteurella multocida, Mycoplasma capricolum, Mycoplasma pneumonia, Mycoplasma hyorhinis, Mycoplasma mycoides, Mannheimia haemolytica, Salmonella enterica, E. coli KTE112, E. coli CFT073, E. coli K-12, Klebsiella pneumonia, Actinobacillus pleuropneumoniae, and Cronobacter sakazakii.

In another aspect, the present invention provides a method of screening a mannitol metabolic inhibitor by detecting activity of an enzyme involved in a mannitol metabolism in vitro.

In one embodiment of the present invention, the enzyme is purified by overexpression, and the enzyme activity is detected by treating a reaction solution of the enzyme with an inhibitor candidate material.

In another embodiment of the present invention, a reaction solution of the enzyme includes fructose-6-phosphate and NADH, or Mtl-1-phosphate and NAD as substrates, and the enzyme activity is estimated by measuring optical density of NADH at 340 nm.

In still another embodiment of the present invention, the enzyme involved in the mannitol metabolism is selected from the group consisting of mannitol-1-phosphate-5-dehydrogenase (M1PDH), mannitol-2-dehydrogenase, mannitol-1-phosphatase, a mannitol repressor, mannitol ABC transporter permease, and mannitol-specific PTS enzyme.

In still another aspect, the present invention provides a method of screening a mannitol metabolic inhibitor by culturing bacteria in the presence of mannitol and measuring viability of the bacteria.

In one embodiment of the present invention, the culturing includes culturing bacteria in a medium containing 55 to 500 mM of mannitol, and the measurement of viability includes treating the culture solution with an inhibitor candidate material and measuring a colony forming unit (CFU) or a concentration of bacteria at OD600.

In yet another aspect, the present invention provides a method of screening a mannitol metabolic inhibitor by measuring a color change of phenol red while bacteria are cultured in the presence of mannitol.

In one embodiment of the present invention, the culturing includes culturing bacteria in a medium including 27 to 500 mM of mannitol and phenol red, and the measurement of the color change includes checking whether the phenol red is or not maintained red.

In yet another aspect, the present invention provides a method of screening a mannitol metabolic inhibitor by measuring viability of bacteria after the bacteria are infected into macrophages in the presence of mannitol.

In another embodiment of the present invention, the bacteria are selected from the group consisting of Staphylococcus aureus, Staphylococcus haemolyticus, Staphylococcus saprophyticus, Streptococcus mutants, Streptococcus pneumoniae, Streptococcus pyogenes, Bacillus anthracis, Pseudomonas aeruginosa, Pseudomonas stutzeri, Vibrio cholera, Vibrio vulnificus, Vibrio parahaemolyticus, Shigella flexneri, Yersinia enterocolitica, Yersinia pesti, Aeromonas salmonicida, Mycoplasma mycoides, Enterococcus faecalis, Yersinia pseudotuberculosis, Pasteurella multocida, Mycoplasma capricolum, Mycoplasma pneumonia, Mycoplasma hyorhinis, Mycoplasma mycoides, Mannheimia haemolytica, Salmonella enterica, E. coli KTE112, E. coli CFT073, E. coli K-12, Klebsiella pneumonia, Actinobacillus pleuropneumoniae, and Cronobacter sakazakii.

In yet another aspect, the present invention provides an antimicrobial composition and cosmetic material containing a mannitol metabolic inhibitor and mannitol as active ingredients.

In one embodiment of the present invention, the mannitol metabolic inhibitor is an inhibitor which is targeted at mannitol 1 phosphate dehydrogenase.

In another embodiment of the present invention, the mannitol metabolic inhibitor is a Mannitol-1-phosphate-5-dehydrogenase (M1PDH) inhibitor.

In still another embodiment of the present invention, the M1 PDH inhibitor is selected from the group consisting of 6-amino-3-methyl-4-(4-nitrophenyl)-1-phenyl-1,4-dihydropyrano[2,3-c]pyrazole-5-carbonitrile, 2-(1-adamantyl)-4-methoxy-6-{[({[2-(trifluoromethyl)phenyl]sulfonyl}amino)carbonyl]amino}-1,3,5-triazine, 3-amino-2-benzyl-7-nitro-4-(2-quinolyl)-1,2-dihydroisoquinolin-1-one, N-[4-(4-chlorophenoxy)-3-nitrobenzoyl]-N′-[2-(trifluoromethyl)phenyl]urea and (2R,4aS,6aS,6aS,14aS,14bR)-10,11-dihydroxy-2,4a,6a, 6a, 9,14a-hexamethyl-3,4,5,6,8,13,14,14b-octahydro-1H-picene-2-carboxylic acid.

In yet another embodiment of the present invention, the composition and cosmetic material have antimicrobial activity against bacteria having mannitol dehydrogenase.

In yet another embodiment of the present invention, the bacteria are selected from the group consisting of Staphylococcus aureus, Staphylococcus haemolyticus, Staphylococcus saprophyticus, Streptococcus mutants, Streptococcus pneumoniae, Streptococcus pyogenes, Bacillus anthracis, Pseudomonas aeruginosa, Pseudomonas stutzeri, Vibrio cholera, Vibrio vulnificus, Vibrio parahaemolyticus, Shigella flexneri, Yersinia enterocolitica, Yersinia pesti, Aeromonas salmonicida, Mycoplasma mycoides, Enterococcus faecalis, Yersinia pseudotuberculosis, Pasteurella multocida, Mycoplasma capricolum, Mycoplasma pneumonia, Mycoplasma hyorhinis, Mycoplasma mycoides, Mannheimia haemolytica, Salmonella enterica, E. coli KTE112, E. coli CFT073, E. coli K-12, Klebsiella pneumonia, Actinobacillus pleuropneumoniae, and Cronobacter sakazakii.

In yet another embodiment of the present invention, the composition and cosmetic material include mannitol at a concentration of 2.74 to 164 mM.

In yet another aspect, the present invention provides an antimicrobial use of a composition containing a mannitol metabolic inhibitor and mannitol as active ingredients.

### BRIEF DESCRIPTION OF THE DRAWINGS

The above and other objects, features, and advantages of the present invention will become more apparent to those of ordinary skill in the art by describing in detail exemplary embodiments thereof with reference to the adhered drawings, in which:

FIGS. 1A-1C show crystal structures of M1PDH using X rays (1.8 Å);

FIG. 2 shows comparison of structures of domains of N-terminals and C-terminals between Staphylococcus aureus M1PDH (hereinafter, referred to as saM1PDH) and Pseudomonas fluorescence mannitol-2-dehydrogenase (pfM2DH) using X rays (1.8 Å);

FIGS. 3A-3D show comparison of structures of a complex (saM1PDH) formed by binding M1PDH with SO4²⁻ and a complex formed by binding pfM2DH and mannitol (MtL) using X rays (1.8 Å);

FIGS. 4A-4B show enzyme activity of M1PDH when residues R283, R287, and R294 are mutated;

FIG. 5A shows activity of M1PDH against mannitol-1-phosphate according to the concentration of NaCl, and FIG. 5B shows activity of M1PDH against fructose-6-phosphate according to the concentration of NaCl;

FIG. 6A shows activity of M1PDH against mannitol-1-phosphate according to the concentration of glycerol, and FIG. 6B shows activity of M1PDH against fructose-6-phosphate according to the concentration of glycerol;

FIG. 7 shows comparison of viability between an M1PDH knock-out mutant (ΔM1PDH) SA (Staphylococcus Aureus) and wild-type (WT) SA under stresses of high concentration of salt and reactive oxygen species (ROS);

FIG. 8A shows death of host cells, which is detected by activity of caspase 3/7 Gb, after an M1PDH knock-out mutant (ΔM1PDH) SA and wild-type (WT) SA are treated with macrophage RAW264.7 host cells, FIG. 8B is a colony forming unit (CFU) of bacteria under the same conditions, and FIG. 8C shows the CFU of bacteria detected by the number of colonies;

FIG. 9A shows a result of an assay for mannitol uptake between an M1PDH knock-out mutant (ΔM1PDH) SA and wild-type (WT) SA, and FIG. 9B shows a degree of bacteriolysis when 27 mM of mannitol is treated for 48-72 hours;

FIG. 10A shows a result of evaluating viability of bacteria after an M1PDH knock-out mutant (ΔM1PDH) SA and wild-type (WT) SA are infected into macrophages in the presence of 2.74 mM of mannitol, and FIG. 10B shows a result of evaluating viability of bacteria in a bacterial culture solution in the presence of 55 mM of mannitol;

FIG. 11A shows a result of confirming a mannitol metabolism inhibitory effect on compounds selected by virtual screening, and FIG. 11B shows an effect of inhibiting growth of bacteria confirmed by a chemical toxicity assay;

FIG. 12A shows a result of performing an in vitro M1PDH enzymatic assay for compounds 10, 26, 27, and 31 (corresponding to Formulas 1, 2, 3, and 4, respectively) in the presence of fructose-6-phosphate or Mtl-1-phosphate, and FIG. 12B shows viability of bacteria infected into microphages in the presence of 2.74 mM of mannitol and cultured after treating 1-200 μM of each compound;

FIG. 13A shows a screening result obtained from natural substance libraries, and FIG. 13B shows inhibitory activity for M1PDH of a compound (Formula 5) ensured through an in vitro assay;

FIG. 14A is a result of confirming viability of bacteria by measuring optical density at 600 nm when the compound of Formula 5 is treated, and FIG. 14B shows a result of proving that the compound of Formula 5 is not directly influenced on host cells by confirming viability of mammalian cells by using a cell viability assays kits, measuring optical density at 450 nm; and

FIG. 15 shows SA viability in macrophages after 18 hours of infection in a macrophage infection model when the compound of Formula 5 and mannitol are used together.

FIG. 16 shows a schematic diagram of a mannitol metabolism pathway.

### DETAILED DESCRIPTION OF EXEMPLARY EMBODIMENTS

Hereinafter, exemplary embodiments of the present invention will be described in detail with reference to examples according to the present invention and comparative examples not according to the present invention. However, the scope of the present invention is not limited to the embodiments to be disclosed below.

The present invention relates to a new antimicrobial method of killing bacteria by lysis through inhibition of a mannitol metabolism, a method of screening an antimicrobial drug, and an antimicrobial composition and cosmetic material containing a mannitol metabolic inhibitor and mannitol as active ingredients.

The mannitol is metabolized by a mannitol operon (mt1A, mt1R, mt1F, or mt1D) composed of four genes. Here, mt1A and mt1F encodes mannitol transport membrane proteins recognizing and phosphorylating extracellular or intracellular mannitol, mt1D encodes a protein causing reversible modification of fructose-6-phosphate and mannitol-1-phosphate, and mt1R encodes a transcription factor whose function is not identified.

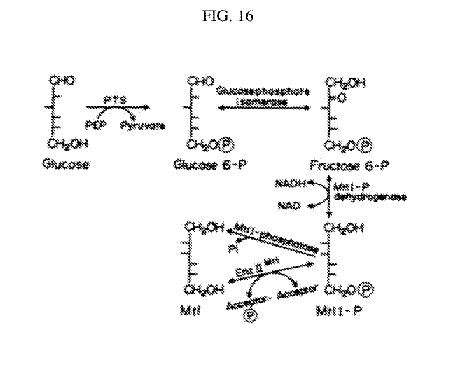

As will be confirmed from the schematic diagram of a mannitol metabolism pathway shown in FIG. 16, M1PDH catalyzes a reversible reaction between mannitol-1-phosphate and fructose-6-phosphate.

An example of the enzyme involved in the mannitol metabolism may be mannitol 2-dehydrogenase, mannitol-1-phosphatase, mannitol-1-phosphate-5-dehydrogenase, a mannitol repressor, mannitol ABC transporter permease, or a mannitol-specific PTS enzyme, and in the present invention, particularly, an antimicrobial effect using an inhibitor targeted at M1PDH was confirmed.

First, a structure of an M1DPH protein is analyzed by crystallization, and particularly, an amino acid residue critical to an enzyme activity is identified and verified by mutation.

In addition, specificity for mannitol-1-phosphate or fructose-6-phosphate substrates according to external osmotic stress or ROS against M1DPH (M1PDH) was confirmed, and based on this, a decrease in viability of SA causing M1PDH knock-out mutation in a high concentration salt-containing medium (14% of NaCl) or an ROS environment (cell-free hydroxyl radical generating system: H₂O₂+FeSO₄+NaI) is confirmed.

In addition, although the mannitol metabolism is blocked, it is confirmed that mannitol is accumulated inside whenever mannitol is present in the bacteria culture environment without being secreted to an outside, and therefore, an antimicrobial effect is confirmed as an osmotic stress environment caused by mannitol accumulation is formed and thus bacteria are killed by lysis by supply of mannitol from an outside of bacteria and inhibition of M1PDH.

In addition, a compound for inhibiting M1PDH activity is screened from a natural compound library by virtual screening according to a docking study, and an inhibitory effect of the compound is verified by performing M1PDH enzymatic assay.

As a result, an M1PDH inhibitor of the present invention may be, but is not limited to, selected from the group consisting of 6-amino-3-methyl-4-(4-nitrophenyl)-1-phenyl-1,4-dihydropyrano[2,3-c]pyrazole-5-carbonitrile, 2-(1-adamantyl)-4-methoxy-6-{[({[2-(trifluoromethyl)phenyl]sulfonyl}amino)carbonyl]amino}-1,3,5-triazine, 3-amino-2-benzyl-7-nitro-4-(2-quinolyl)-1,2-dihydroisoquinolin-1-one, N-[4-(4-chlorophenoxy)-3-nitrobenzoyl]-N′-[2-(trifluoromethyl)phenyl]urea, and (2R,4aS,6aS,6aS,14aS,14bR)-10,11-dihydroxy-2,4a,6a,6a,9,14a-hexamethyl-3,4,5,6,8,13,14,14b-octahydro-1H-picene-2-carboxylic acid.

In addition, the antimicrobial composition of the present invention may have antimicrobial activity against bacteria involved in a mannitol metabolism, which may be, but is not limited to, Staphylococcus aureus, Staphylococcus haemolyticus, Staphylococcus saprophyticus, Streptococcus mutants, Streptococcus pneumoniae, Streptococcus pyogenes, Bacillus anthracis, Pseudomonas aeruginosa, Pseudomonas stutzeri, Vibrio cholera, Vibrio vulnificus, Vibrio parahaemolyticus, Shigella flexneri, Yersinia enterocolitica, Yersinia pesti, Aeromonas salmonicida, Mycoplasma mycoides, Enterococcus faecalis, Yersinia pseudotuberculosis, Pasteurella multocida, Mycoplasma capricolum, Mycoplasma pneumonia, Mycoplasma hyorhinis, Mycoplasma mycoides, Mannheimia haemolytica, Salmonella enterica, E. coli KTE112, E. coli CFT073, E. coli K-12, Klebsiella pneumonia, Actinobacillus pleuropneumoniae, and Cronobacter sakazakii.

The term “antimicrobial activity” used herein refers to a capability of resisting to bacteria, and includes all of mechanisms occurring to protected from an action of microorganisms such as bacteria, fungi, and yeast.

According to an embodiment of the present invention, the antimicrobial composition may be prepared as a pharmaceutical composition.

The pharmaceutical composition contains mannitol and a mannitol metabolic inhibitor as active ingredients, and may include a pharmaceutically available carrier. The pharmaceutically available carrier is conventionally used to preparation, includes, but is not limited to, saline, sterilized water, a Ringer's solution, buffered saline, cyclodextrin, a dextrose solution, a maltodextrin solution, glycerol, ethanol, or liposome, and when needed, may further include another conventional additive such as an antioxidant or a buffer solution. In addition, the pharmaceutical composition may be prepared as an injection form such as an aqueous solution, a suspension, or an emulsion, a pill, a capsule, a granule, or a tablet by adding a diluent, a dispersant, a surfactant, a bonding agent, or a lubricant. In terms of a suitable pharmaceutically available carrier and preparation, the preparation may be performed by a method disclosed in Remington's Pharmaceutical Science, Mack Publishing Company, Easton Pa. according to a component. The pharmaceutical composition of the present invention may be prepared as an injection, an inhalant, or an external application to skin, but a dosage form thereof is not particularly limited.

A method of administering a pharmaceutical composition of the present invention may be, but is not particularly limited to, parenteral administration such as intravenous administration, subcutaneous administration, intraperitoneal administration, inhalation, skin application, or topical application, or oral administration according to a desired method.

A dose varies depending on a weight, age, sex, health condition, diet, administration time, administration method, an excretion rate, and severity of a disease. A daily dose refers to an amount of a therapeutic material of the present invention sufficient for treatment with respect to a relieved state of a disease by administering an individual required to be treated. An effective amount of the therapeutic material varies depending on a specific compound, a condition of a disease, severity of a disease, and individuals required to be treated, and may be conventionally determined by those of ordinary skill in the art. As a non-limited example, a dose for a human body of the composition according to the present invention may vary depending on a patient's age, a body weight, an administration type, a health condition, and a condition of a disease, and based on a 70-kg adult patient, generally a dose is 0.01 to 1,000 mg/day, and preferably 1 to 500 mg/day. The composition may be administered once to several times a day at a predetermined interval.

In the composition of the present invention, an amount of mannitol may be included at 2.74 to 164 mM, but the present invention is not limited thereto. In the example of the present invention, for the M1PDH knock-out mutation, the minimum concentration is determined based on bacteriolysis in the presence of 2.74 mM or higher of mannitol, and the maximum content is determined based on a content of mannitol clinically used.

Since mannitol needs a long-term metabolism in a body, and is retained for a long time in blood in the form of a polysaccharide not permeating through a blood-brain barrier, the mannitol is used to treat acute renal failure or brain oedema. A 15%, 20%, or 25% solution is prepared by diluting 1 to 3 g of mannitol per kg of a body weight, and usually administered once a day by drip infusion, and a daily maximum dose is limited to 200 g. As described above, since the mannitol currently used as various types of a therapeutic agent may be used in combination with the mannitol metabolic inhibitor, the composition may be used as complicated prescriptions for various diseases.

In one embodiment of the present invention, the antimicrobial composition may be prepared as a cosmetic composition.

The cosmetic composition may contain mannitol and a mannitol inhibitor as active ingredients, and include components conventionally used in a cosmetic composition. For example, the cosmetic composition may include a conventional adjuvant such as an antioxidant, a stabilizer, a solubilizer, a vitamin, a pigment, or a fragrance, and/or a carrier.

When the antimicrobial composition of the present invention containing mannitol and a mannitol inhibitor as active ingredients is used as a cosmetic additive, the composition may be added or used with another cosmetic component, and may be suitably used according to a conventional method. A mixed amount of the active ingredient may be suitably determined according to a purpose of use (prevention, health, or therapeutic treatment).

The cosmetic composition of the present invention may be prepared in any dosage form conventionally prepared in the art, for example, a solution, a suspension, an emulsion, a paste, a gel, a cream, a lotion, powder, soap, a surfactant-containing cleanser, oil, a powder foundation, an emulsion foundation, a wax foundation, and a spray, but the present invention is not limited thereto. More specifically, the composition may be prepared in a dosage form such as a toner, an emulsifier, a cream, a nutrient cream, a massage cream, an essence, an eye cream, a cleansing cream, a cleansing foam, a cleansing water, a pack, a spray, or a powder.

When the dosage form of the present invention is a paste, cream, or gel, as a carrier component, animal oil, vegetable oil, wax, paraffin, starch, tragacanth, a cellulose derivative, polyethylene glycol, silicon, bentonite, silica, talc, or zinc oxide may be used.

When the dosage form of the present invention is a powder or spray, as a carrier component, lactose, talc, silica, aluminum hydroxide, calcium silicate, or polyamide powder may be used, and particularly when the dosage form of the present invention is a spray, a propellant such as chlorofluorohydrocarbon, propane/butane, or dimethyl ether may be further included.

In addition, when the dosage form of the present invention is a solution or emulsion, as a carrier component, a solvent, a solubilizer or an emulsifier, particularly, water, ethanol, isopropanol, ethyl carbonate, ethyl acetate, benzyl alcohol, benzyl benzoate, propylene glycol, 1,3-butylglycol oil, glycerol aliphatic ester, polyethylene glycol, or aliphatic ester of sorbitan, may be used.

In addition, when the dosage form of the present invention is a suspension, as a carrier component, a liquid-type diluent such as water, ethanol, or propylene glycol, a suspending agent such as ethoxylated isostearyl alcohol, polyoxyethylene sorbitol ester, and polyoxyethylene sorbitan ester, crystallite cellulose, aluminum metahydroxide, bentonite, agar, or tragacanth may be used.

In addition, when the dosage form of the present invention is a interface-active agent-containing cleaning agent, as a carrier component, aliphatic alcohol sulfate, aliphatic alcohol ether sulfate, sulfosuccinic acid monoester, isethionate, an imidazolinium derivative, methyltaurate, sarcosinate, fatty acid amide ether sulfate, alkylamidobetaine, aliphatic alcohol, fatty acid glyceride, fatty acid diethanolamide, vegetable oil, a lanolin derivative, or ethoxylated glycerol fatty acid ester may be used.

In the cosmetic composition of the present invention, 2.74 to 164 mM of mannitol may be included, but the present invention is not limited thereto. In the example of the present invention, for the M1PDH knock-out mutation, the minimum concentration is determined based on bacteriolysis in the presence of 2.74 mM or higher of mannitol, and the maximum content is determined based on a content of mannitol clinically used today.

In an embodiment of the present invention, an antimicrobial method for inhibiting a mannitol metabolism including treating bacteria with a mannitol dehydrogenase inhibitor and 2.74 to 164 mM of mannitol is provided.

In this method, the mannitol dehydrogenase becoming a target may be, but is not limited to, mannitol 2-dehydrogenase, mannitol-1-phosphatase, mannitol ABC transporter permease, mannitol-specific PTS enzyme, or M1PDH.

In addition, the method is performed in vitro, but the present invention is not limited thereto.

Hereinafter, an exemplary example is provided to help understanding of the present invention. However, the following examples are merely provided to more easily understand the present invention, not to limit the scope of the present invention.

### Example 1

### Confirmation of Structure of Crystallized M1PDH

To understand an enzymatic mechanism of M1PDH, M1PDH was crystallized, and a three-dimensional structure of the enzyme was finally identified by solving a phase problem using a 1.8 Å X-ray diffraction data obtained from the M1PDH crystal through molecular replacement. In addition, structures of N-terminal and C-terminal domains and active sites of M1PDH and pfM2DH (pseudomonas fluorescence mannitol 2-dehydrogenase) were compared.

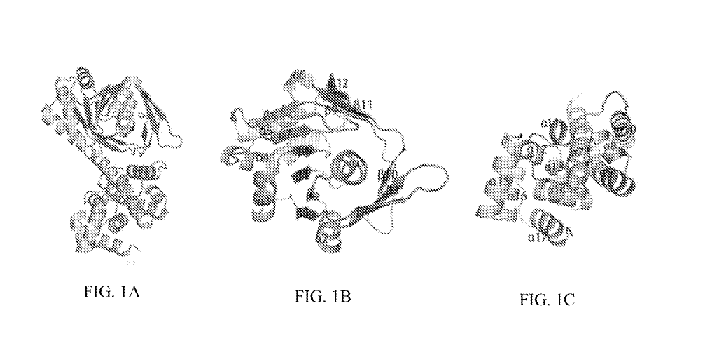

As a result, as shown in FIG. 1, M1PDH is composed of a monomer, and separated into a C-terminal domain and an N-terminal domain. It was confirmed that the N-terminal domain (residue 1-190) formed a Rossmann fold typically shown in a nucleic acid-binding protein (FIG. 1A), both sides of the fold were composed of five α-helix (α1, α2, α3, α4, and α5) structures, and a center of the fold was composed of six parallel β-sheets (β5, β2, β1, β6, β7, and β8 in FIG. 1B). In addition, it was confirmed that the C-terminal domain (residue 190-368) was composed of eleven α-helix structures (FIG. 1C). Here, a first sheet (β9, β11, or β12) was linked to the C-terminal, and a second sheet (β10, β3, or β4) was linked to the N-terminal.

### Example 2

### Confirmation of Residue Specific to M1PDH Activity

2-1: Comparison of Structures of M1PDH and pfM2DH

To detect an enzymatic mechanism and an active site of M1PDH, structures of N-terminal and C-terminal domains and active sites of M1 PDH and pfM2DH were compared.

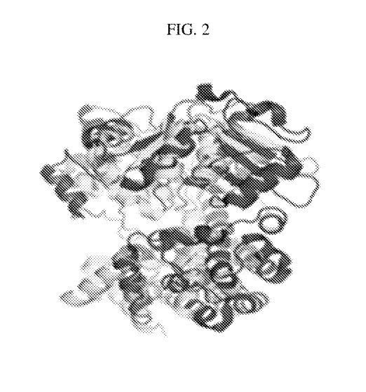

As a result, as shown in FIG. 2, all of M1PDH and pfM2DH had similar core regions including two β-sheets and Rossmann folding, but compared with the structure of pfM2DH, in the structure of M1PDH, an external helix structure was less developed, and an active site pocket between domains had a larger structure.

2-2: Comparison of Structures of saM1PDH and afM2DH

In addition, as a structure of a complex (saM1PDH) formed by binding M1PDH with SO4²⁻ was compared with a structure of a complex (afM2DH) formed by binding pfM2DH with mannitol (MtL), specificity of M1PDH with respect to a mannitol-1-phosphate or fructose-6-phosphate substrate was confirmed.

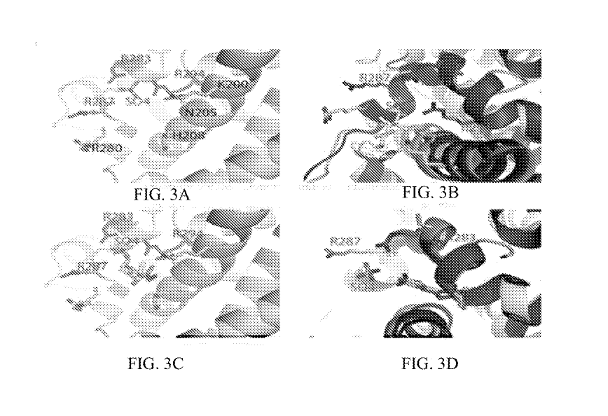

As a result, as shown in FIG. 3, in the saM1PDH structure, SO4²⁻ was bound to an R283, R287, or R294 residue of M1PDH, and it was confirmed that these residues are highly-conserved in the active site of other related proteins.

Actually, as a result that the mannitol-1-phosphate or fructose-6-phosphate substrate was subjected to manual docking to M1PDH, SO4²⁻ well overlapped PO4²⁻ of the mannitol-1-phosphate or fructose-6-phosphate substrate. Accordingly, it was seen that the residues R283, R287, and R294 were residues critical to enzyme specificity of M1PDH.

2-3: Activity of M1PDH Mutant

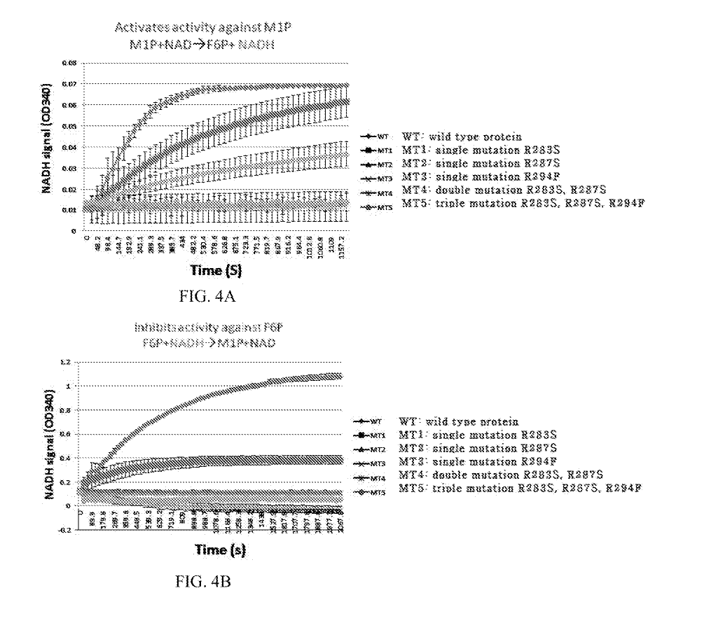

To confirm the result of Example 2-2 in further detail, when the residues R283, R287, and R294 corresponding to PO4²⁻ binding sites were mutated, production of NADH in a reaction of producing fructose-6-phosphate from mannitol-1-phosphate (FIG. 4A) and loss of NADH in a reaction producing mannitol-1-phosphate from fructose-6-phosphate (FIG. 4B) were measured at 340 nm, thereby measuring an enzyme activity of M1PDH.

That is, as a result of analyzing an enzyme activity against mannitol-1-phosphate and fructose-6-phosphate substrates by modifying wild-type residues R283, R287, and R294 to R283S, R287S, and R294F, as shown in FIG. 4, the enzyme activity of M1PDH with modified R283 and R287 was significantly decreased, and there was almost no enzyme activity of M1PDH with modified R294. Accordingly, it was clear that the residues R283, R287, and R294 were important in activity of the M1PDH enzyme.

### Example 3

### Substrate Specificity of M1PDH According to External Stress

According to analysis of Staphylococcus aureus(SA) genomes, M1PDH is known as the only one enzyme involved in a reversible reaction converting mannitol-1-phosphate to fructose-6-phosphate, or fructose-6-phosphate to mannitol-1-phosphate. In addition, mannitol is a polar material which is synthesized in a cell, or input from an outside of the cell, and a compound scavenging a hydroxyl radical.

Accordingly, in this example, the following experiment was performed on the assumption that M1PDH would serve as a critical role to a mannitol metabolism in an environment having mannitol as an enzyme reacting to stress such as osmosis.

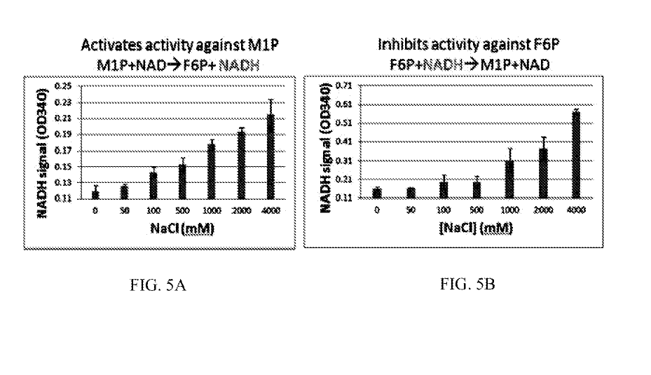

That is, based on that selective substrate specificity of M1PDH to mannitol-1-phosphate or fructose-6-phosphate could be determined according to a level of osmotic stress or ROS, production of NADH in a reaction of producing fructose-6-phosphate from mannitol-1-phosphate (FIG. 5A) and loss of NADH in a reaction of producing mannitol-1-phosphate from fructose-6-phosphate (FIG. 5B) were measured at 340 nm, thereby measuring a concentration of NADH, and thus substrate specificity of M1PDH obtained by treatment of NaCl or glycerol causing osmotic stress by concentration was compared.

As a result, as shown in FIG. 5, while activity of M1PDH converting mannitol-1-phosphate to fructose-6-phosphate (FIG. 5A) was increased, as the concentration of NaCl was increased, the activity of M1PDH converting fructose-6-phosphate to mannitol-1-phosphate (FIG. 5B) was decreased.

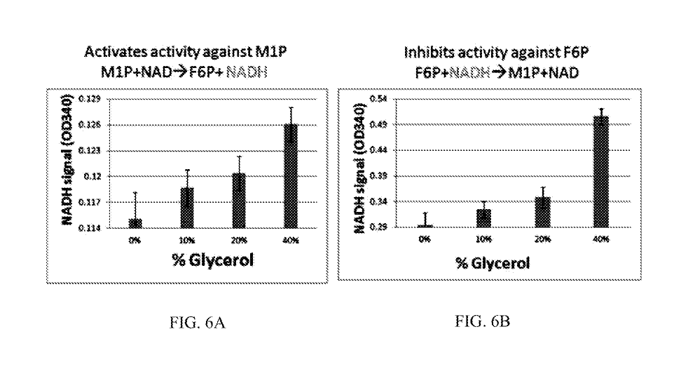

In addition, as shown in FIG. 6, while the activity of M1PDH converting mannitol-1-phosphate to fructose-6-phosphate (FIG. 6A) was increased according to the increase in glycerol concentration, the activity of M1PDH converting fructose-6-phosphate to mannitol-1-phosphate (FIG. 6B) was decreased.

Accordingly, it was seen that the substrate specificity of M1PDH was adjusted by osmotic stress. That is, in a hypertonic environment, the form of M1PDH was changed to convert mannitol-1-phosphate into fructose-6-phosphate, and as a result, a concentration of the entire solvent in the cell was decreased to prevent the lysis of bacterial cells caused by the input of moisture. Contrarily, in a hypotonic environment, the form of M1PDH was changed to convert fructose-6-phosphate into mannitol-1-phosphate, and as a result, a concentration of the entire solvent in the cell was increased to prevent contraction of the cell by loss of moisture from the cell.

### Example 4

### Evaluation of SA Viability in M1PDH Knock-Out Mutants

4-1: Cell Viability Under Stresses of High Concentration Salt and ROS

Mannitol is metabolized by SA and synthesized in a cell (to be a glucose metabolic product), but a function of mannitol in bacteria is not clearly known. On the assumption that the uptake of mannitol from an outside of the cell or synthesis of mannitol in the cell would similarity work to cause toxicity and a stress in bacterial infection, in this example, the following experiment was performed.

That is, when metabolism of mannitol was inhibited by the knock-out of M1PDH, a critical enzyme in a mannitol metabolic pathway, through insertion mutation, SA viability in a high concentration salt (14% NaCl)-containing medium or ROS environment (cell-free hydroxyl radical-generating system: H₂O₂+FeSO₄+NaI) was investigated.

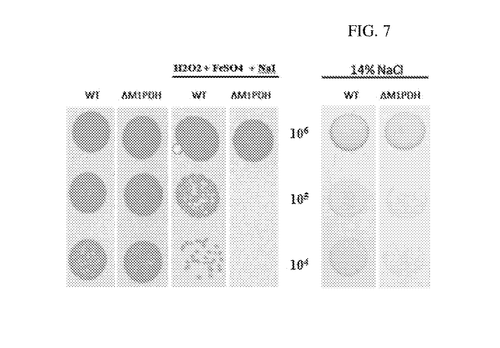

As a result, as shown in FIG. 7, it was confirmed that the M1PDH-knocked-out mutation SA had inhibited synthesis and metabolism of mannitol in all of the high concentration salt and ROS stress environments, compared to the wild type (WT).

4-2: Viability in In Vitro Infection

Since bacteria are placed in various osmosis and ROS stress environments by a host cell defense system in infection, on the assumption that viability of M1PDH knock-out mutant strains would also be decreased by inhibition of the mannitol metabolism in infection, the following experiment was performed.

That is, after M1PDH knock-out mutant SA and wild-type SA were infected using macrophage RAW264.7 of a mouse as a host cell, pathogenicity caused by a death rate of the host cell (RAW264.7) was evaluated using an apoptosis detection kit, and bacteria viability was evaluated. Death of host cells was confirmed by measuring activity of a caspase increasing expression of the host cells during apoptosis, and the viability of the bacteria was confirmed by a colony forming unit (CFU).

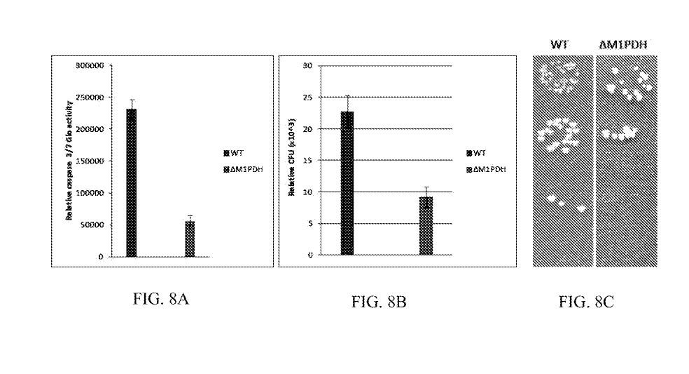

As a result, as shown in FIG. 8, the M1PDH knock-out mutant was decreased in caspase 3/7 Gb activity (FIG. 8A), compared to the wild type (WT), which means that the death of the host cells was reduced, and a decrease in pathogenicity of bacteria was confirmed. In addition, compared to the wild type (WT), it was confirmed that the cell viability of bacteria in the M1PDH knock-out mutants was decreased (FIGS. 8B and 8C).

### Example 5

### Bacterial Death Effect Caused by Mannitol Accumulation of M1PDH Knock-Out Mutants

5-1: Confirmation of Mannitol Accumulation of M1PDH Mutants

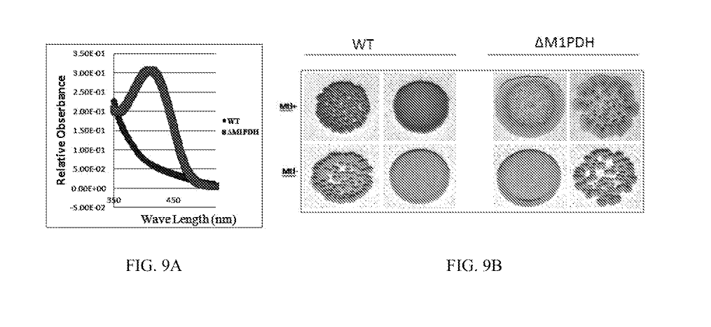

While it is known that mannitol is synthesized in bacteria or uptaken from an outside, it is not known whether mannitol is secreted to an outside when a mannitol metabolism is blocked. To confirm this, M1PDH knock-out mutant SA and wild-type SA were cultured in solid media containing mannitol, and subjected to an assay for mannitol uptake. That is, after mannitol was treated to the medium, mannitol contents in the wild type and the mutant bacteria were measured to confirm a degree of accumulation of mannitol (FIG. 9A), and the influence of mannitol on the existence of bacteria was measured by counting the number of colonies of the wild type and the mutants in the presence of 27 mM of mannitol (FIG. 9B).

As a result, as shown in FIG. 9, unlike the wild type (WT_SA), the M1PDH-knock-out mutant SA was continuously increased in the amount of mannitol, and it was confirmed that bacteria were exploded by osmotic stress caused by accumulation of mannitol in a late stationary phase. Therefore, it was seen that even though the mannitol metabolism was blocked, the mannitol was not secreted to an outside, but continuously accumulated inside.

5-2: Confirmation of Decrease in Pathogenicity of M1PDH Mutant on Mannitol-Containing Medium

To develop a tool for killing SA from the result of Example 5-1, SA viability in the presence of mannitol was evaluated in each of a macrophage infection model and a bacteria medium. That is, SA viability was evaluated when M1PDH-knock-out mutant SA and wild-type SA were infected to macrophages in the presence of 2.74 mM of mannitol, and when M1PDH-knock-out mutant SA and wild-type SA were incubated in 55 mM of a mannitol-containing bacterial medium.

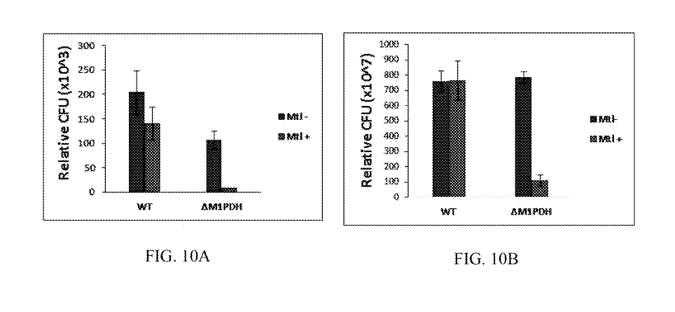

As a result, as shown in FIG. 10, the knock-out mutant SA was significantly decreased in viability, compared to the wild type, and it was seen from the result that when the mannitol metabolism in the bacteria was blocked, and mannitol was provided to an outside of the bacteria, mannitol was uptaken but accumulated without being metabolized, thereby causing osmotic stress, and thus the bacteria were lyzed by the host cell protection system.

### Example 6

### Screening of M1PDH Inhibiting Compound

6-1: Virtual Screening

To screen a compound inhibiting M1PDH activity, virtual screening was performed according to a docking study. The virtual screening is one of structure-based ligand designs, which is a method of screening a material capable of adjusting activity by the bonding to a protein, or a screening method by docking a database of a large scale of chemical libraries to a target receptor using a computer.

In this example, first, virtual screening was performed with respect to approximately 60,000 types of compounds of a commercialized compound library, which is Maybridge chemical library database through the above-described virtual screening to screen 93 types of compounds expected to have an excellent binding strength to active sites of M1PDH (a mannitol binding site and a phosphate group-binding site).

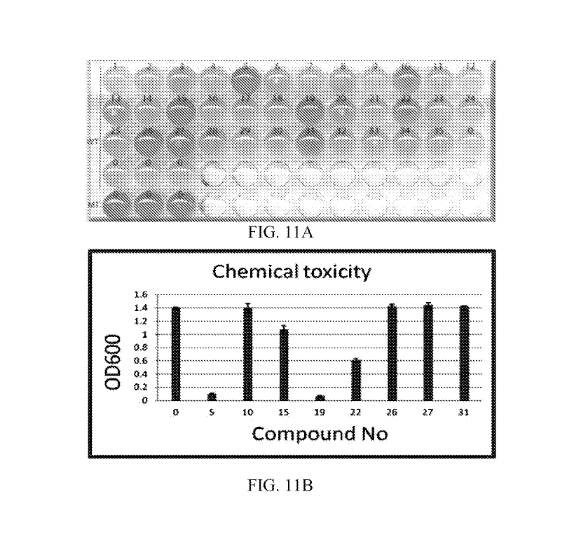

Secondly, 8 types of hit ligands exhibiting a mannitol metabolism inhibitory effect on the above compounds were found (FIG. 11A). When bacteria were cultured in a medium containing 27 mM of mannitol and phenol red, the mannitol was metabolized to produce fructose and reduce pH of the medium, thereby changing a color of the phenol red to yellow. However, since the phenol red was not changed in color to keep the medium red by inhibiting the mannitol metabolism, an inhibitor showing an mannitol metabolism inhibitory effect was found using such a principle (FIG. 11A).

In addition, 4 types of compounds 10, 26, 27, and 31 not inhibiting growth of bacteria but inhibiting only the mannitol metabolism were finally selected by a chemical toxicity assay (FIG. 11B).

6-2: M1PDH Enzymatic Assay

An actual M1PDH inhibitory effect was finally confirmed by performing the M1PDH enzymatic assay to 4 types of the compounds finally selected in Example 6-1.

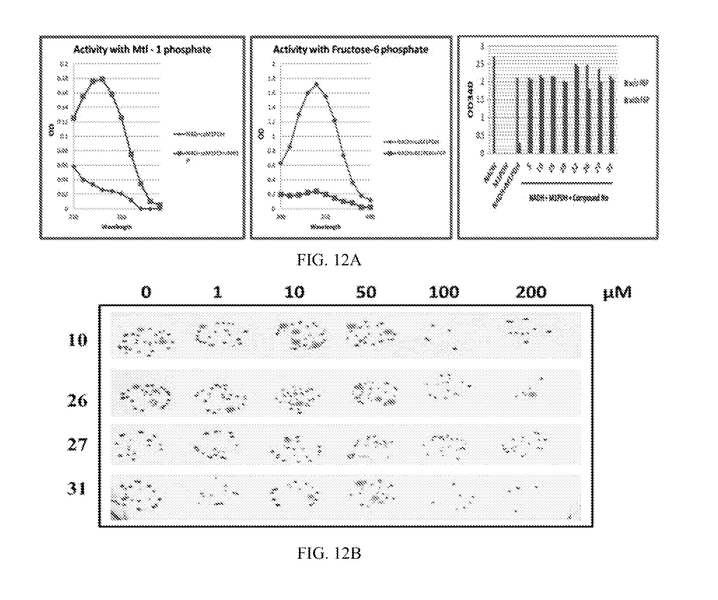

To measure an in vitro M1PDH enzyme activity, a change in concentration of NADH according to an enzyme activity was observed through a change in optical density at 340 nm using fructose-6-phosphate and NADH, or Mtl-1-phosphate and NAD as substrates. As a result, as shown in FIG. 12A, it was confirmed that all of the 4 types of compounds 10, 26, 27, and 31 in Example 6-1 inhibited M1PDH activity.

In addition, to confirm an inhibitory effect in a host cell, a macrophage infection model was used. That is, as shown in FIG. 12B, it was confirmed that the 4 types of the compounds reduced viability of bacteria cultured by treating macrophages with mannitol (2.74 mM) and the 4 types of the compounds at different concentrations from each other.

The 4 types of the compounds 10, 26, 27, and 31 having an inhibitory activity against M1PDH according to the example are as follows:

### 1) 6-amino-3-methyl-4-(4-nitrophenyl)-1-phenyl-1,4-dihydropyrano[2,3-c]pyrazole-5-carbonitrile

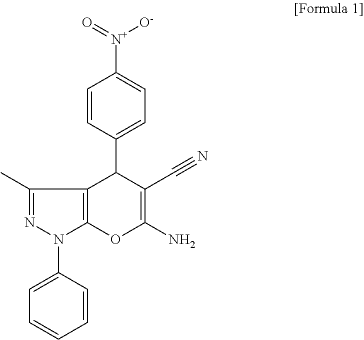 [SMILES: Cc1nn(-c2ccccc2)c2c1C(c1ccc([N+](=O)[O-])cc1)C(C#N)=C(N)O2]

### 2) 2-(1-adamantyl)-4-methoxy-6-{[({[2-(trifluoromethyl)phenyl]sulfonyl}amino)carbonyl]amino}-1,3,5-triazine

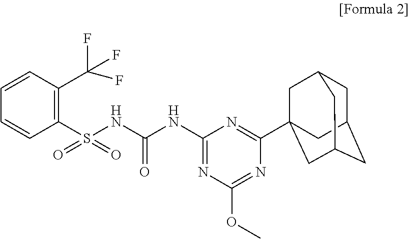 [SMILES: COc1nc(NC(=O)NS(=O)(=O)c2ccccc2C(F)(F)F)nc(C23CC4CC(CC(C4)C2)C3)n1]

### 3) 3-amino-2-benzyl-7-nitro-4-(2-quinolyl)-1,2-dihydroisoquinolin-1-one

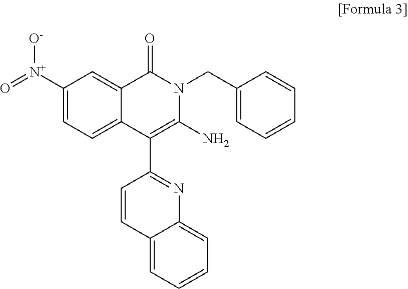 [SMILES: Nc1c(-c2ccc3ccccc3n2)c2ccc([N+](=O)[O-])cc2c(=O)n1Cc1ccccc1]

### 4) N-[4-(4-chlorophenoxy)-3-nitrobenzoyl]-N′-[2-(trifluoromethyl)phenyl]urea

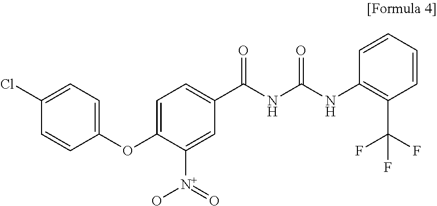

6-3: Screening of Inhibitor Using Natural Compound Library

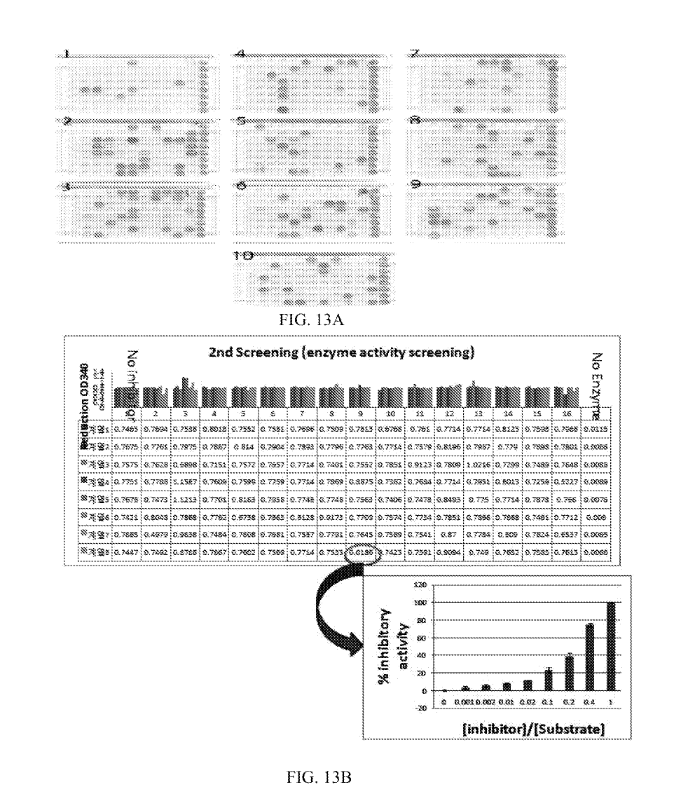

An inhibitor was primarily selected by confirming whether or not to inhibit the mannitol metabolism by treating cells in a medium containing mannitol with a natural compound library (800 types produced by Microsource) (FIG. 13A), and then compound 5 inhibiting an activity of M1PDH as described in Example 6-2 was finally identified from the compounds (FIG. 13B).

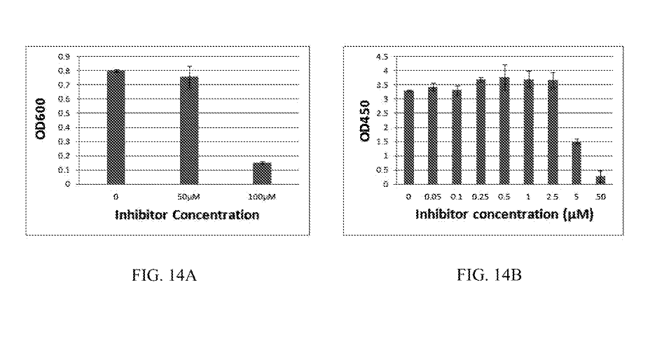

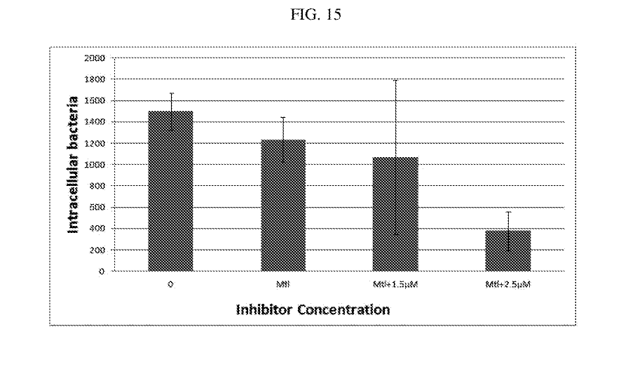

In addition, a decrease in viability of bacteria and mammalian cells caused by the change in concentration of the compound 5 (FIGS. 14A and 14B) was confirmed, and a decrease in SA viability in a macrophage in combination with the compound 5 and mannitol was confirmed using a macrophage infection model to confirm an inhibitory effect in host cells (FIG. 15).

The compound 5 having an inhibitory activity against M1PDH according to the example is as follows:

### 5) (2R,4aS,6aS,6aS,14aS,14bR)-10,11-dihydroxy-2,4a,6a,6a,9,14a-hexamethyl-3,4,5,6,8,13,14,14b-octahydro-1H-picene-2-carboxylic acid

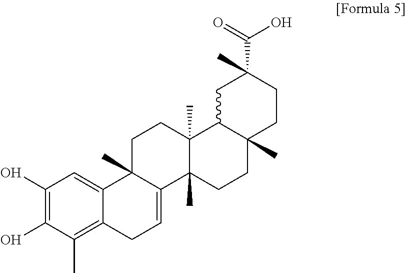 [SMILES: Cc1c(O)c(O)cc2c1CC=C1[C@@]2(C)CC[C@@]2(C)C3C[C@](C)(C(=O)O)CC[C@]3(C)CC[C@]12C]

While the invention has been shown and described with reference to certain exemplary embodiments thereof, it will be understood by those skilled in the related art that various changes in form and details may be made therein without departing from the scope of the invention as defined by the appended claims.

The present invention provides a new method of screening a compound inhibiting a mannitol metabolism, and a new antimicrobial method effectively killing bacteria using a found inhibitor.

Accordingly, the present invention also provides an antimicrobial method and an antimicrobial composition killing bacteria by lysis through inhibition of the mannitol metabolism, unlike conventional antibiotics, and the composition solves a problem of antibiotic resistance and has an excellent antimicrobial effect. Therefore, the composition is expected to be useful for an antimicrobial use.

1. An antimicrobial method of killing bacteria having a mannitol metabolic pathway by inhibiting a mannitol metabolism.

2. The method according to claim 1, which includes treating the bacteria with a mannitol metabolic inhibitor and 55 to 500 mM of mannitol.

3. The method according to claim 2, wherein the treatment is performed in vitro.

4. The method according to claim 1, wherein the bacteria having a mannitol metabolic pathway are selected from the group consisting of Staphylococcus aureus, Staphylococcus haemolyticus, Staphylococcus saprophyticus, Streptococcus mutants, Streptococcus pneumoniae, Streptococcus pyogenes, Bacillus anthracis, Pseudomonas aeruginosa, Pseudomonas stutzeri, Vibrio cholera, Vibrio vulnificus, Vibrio parahaemolyticus, Shigella flexneri, Yersinia enterocolitica, Yersinia pesti, Aeromonas salmonicida, Mycoplasma mycoides, Enterococcus faecalis, Yersinia pseudotuberculosis, Pasteurella multocida, Mycoplasma capricolum, Mycoplasma pneumonia, Mycoplasma hyorhinis, Mycoplasma mycoides, Mannheimia haemolytica, Salmonella enterica, E. coli KTE112, E. coli CFT073, E. coli K-12, Klebsiella pneumonia, Actinobacillus pleuropneumoniae, and Cronobacter sakazakii.

5. A method of screening a mannitol metabolic inhibitor by measuring activity of an enzyme involved in a mannitol metabolism in vitro.

6. The method according to claim 5, wherein the enzyme is purified by overexpression, and the enzyme activity is measured by treating a reaction solution of the enzyme with an inhibitor candidate material.

7. The method according to claim 6, wherein the reaction solution of the enzyme includes fructose-6-phosphate and NADH, or Mtl-1-phosphate and NAD as substrates, and the enzyme activity is estimated by measuring optical density of NADH at 340 nm.

8. The method according to claim 5, wherein the enzyme involved in the mannitol metabolism is selected from the group consisting of mannitol-1-phosphate-5-dehydrogenase (M1PDH), mannitol-2-dehydrogenase, mannitol-1-phosphatase, a mannitol repressor, mannitol ABC transporter permease, and mannitol-specific PTS enzyme.

9. A method of screening a mannitol metabolic inhibitor by culturing bacteria in the presence of mannitol and measuring the bacteria.

10. The method according to claim 9, wherein the measuring is measuring viability of bacteria and the culturing includes culturing bacteria in a medium containing 55 to 500 mM of mannitol, and the measurement of viability includes treating the culture solution with an inhibitor candidate material and measuring a colony forming unit (CFU) or a concentration of bacteria at OD600.

11. The method according to claim 9, wherein the measuring is measuring a color change of phenol red.

12. The method according to claim 11, wherein the culturing includes culturing bacteria in a medium including 27 to 500 mM of mannitol and phenol red, and the measurement of the color change includes checking whether the phenol red is or not maintained red.

13. The method according to claim 9, wherein the measuring is measuring viability of bacteria after the bacteria are infected into macrophages in the presence of mannitol.

14. The method according to claim 13, wherein the culturing includes culturing bacteria in a medium including 2.74 to 164 mM of mannitol, and the viability measurement includes measuring a CFU of the bacteria after a culture solution is treated with an inhibitor candidate material.

15. The method according to claim 9, wherein the bacteria are selected from the group consisting of Staphylococcus aureus, Staphylococcus haemolyticus, Staphylococcus saprophyticus, Streptococcus mutants, Streptococcus pneumoniae, Streptococcus pyogenes, Bacillus anthracis, Pseudomonas aeruginosa, Pseudomonas stutzeri, Vibrio cholera, Vibrio vulnificus, Vibrio parahaemolyticus, Shigella flexneri, Yersinia enterocolitica, Yersinia pesti, Aeromonas salmonicida, Mycoplasma mycoides, Enterococcus faecalis, Yersinia pseudotuberculosis, Pasteurella multocida, Mycoplasma capricolum, Mycoplasma pneumonia, Mycoplasma hyorhinis, Mycoplasma mycoides, Mannheimia haemolytica, Salmonella enterica, E. coli KTE112, E. coli CFT073, E. coli K-12, Klebsiella pneumonia, Actinobacillus pleuropneumoniae, and Cronobacter sakazakii.

16. An antimicrobial composition containing a mannitol metabolic inhibitor and mannitol as active ingredients.

17. The composition according to claim 16, wherein the mannitol metabolic inhibitor is an inhibitor targeted at mannitol dehydrogenase.

18. The composition according to claim 16, wherein the mannitol metabolic inhibitor is a Mannitol-1-phosphate-5-dehydrogenase (M1PDH) inhibitor.

19. The composition according to claim 18, wherein the M1PDH inhibitor is selected from the group consisting of 6-amino-3-methyl-4-(4-nitrophenyl)-1-phenyl-1,4-dihydropyrano[2,3-c]pyrazole-5-carbonitrile, 2-(1-adamantyl)-4-methoxy-6-{[({[2-(trifluoromethyl)phenyl]sulfonyl}amino)carbonyl]amino}-1,3,5-triazine, 3-amino-2-benzyl-7-nitro-4-(2-quinolyl)-1,2-dihydroisoquinolin-1-one, N-[4-(4-chlorophenoxy)-3-nitrobenzoyl]-N′-[2-(trifluoromethyl)phenyl]urea, and (2R,4aS,6aS,6aS,14aS,14bR)-10,11-dihydroxy-2,4a,6a,6a,9,14a-hexamethyl-3,4,5,6,8,13,14,14b-octahydro-1H-picene-2-carboxylic acid.

20. The composition according to claim 16, which has antimicrobial activity against bacteria having mannitol metabolizing enzyme.

21. The composition according to claim 20, wherein the bacteria are selected from the group consisting of Staphylococcus aureus, Staphylococcus haemolyticus, Staphylococcus saprophyticus, Streptococcus mutants, Streptococcus pneumoniae, Streptococcus pyogenes, Bacillus anthracis, Pseudomonas aeruginosa, Pseudomonas stutzeri, Vibrio cholera, Vibrio vulnificus, Vibrio parahaemolyticus, Shigella flexneri, Yersinia enterocolitica, Yersinia pesti, Aeromonas salmonicida, Mycoplasma mycoides, Enterococcus faecalis, Yersinia pseudotuberculosis, Pasteurella multocida, Mycoplasma capricolum, Mycoplasma pneumonia, Mycoplasma hyorhinis, Mycoplasma mycoides, Mannheimia haemolytica, Salmonella enterica, E. coli KTE112, E. coli CFT073, E. coli K-12, Klebsiella pneumonia, Actinobacillus pleuropneumoniae, and Cronobacter sakazakii.

22. The composition according to claim 16, which includes mannitol at a concentration of 2.74 to 164 mM.

23. An antimicrobial cosmetic material comprising a mannitol metabolic inhibitor and mannitol as active ingredients.

24. The cosmetic material according to claim 23, wherein the mannitol metabolic inhibitor is an inhibitor targeted at mannitol metabolizing enzyme.

25. The cosmetic material according to claim 24, wherein the mannitol metabolic inhibitor is a Mannitol-1-phosphate-5-dehydrogenase (M1PDH) inhibitor.

26. The cosmetic material according to claim 25, wherein the M1PDH inhibitor is selected from the group consisting of 6-amino-3-methyl-4-(4-nitrophenyl)-1-phenyl-1,4-dihydropyrano[2,3-c]pyrazole-5-carbonitrile, 2-(1-adamantyl)-4-methoxy-6-{[({[2-(trifluoromethyl)phenyl]sulfonyl}amino)carbonyl]amino}-1,3,5-triazine, 3-amino-2-benzyl-7-nitro-4-(2-quinolyl)-1,2-dihydroisoquinolin-1-one, N-[4-(4-chlorophenoxy)-3-nitrobenzoyl]-N′-[2-(trifluoromethyl)phenyl]urea, and (2R,4aS,6aS,6aS,14aS,14bR)-10,11-dihydroxy-2,4a,6a,6a,9,14a-hexamethyl-3,4,5,6,8,13,14,14b-octahydro-1H-picene-2-carboxylic acid.

27. The cosmetic material according to claim 23, which has an antimicrobial activity against bacteria having mannitol metabolizing enzyme.

28. The cosmetic material according to claim 27, wherein the bacteria are selected from the group consisting of Staphylococcus aureus, Staphylococcus haemolyticus, Staphylococcus saprophyticus, Streptococcus mutants, Streptococcus pneumoniae, Streptococcus pyogenes, Bacillus anthracis, Pseudomonas aeruginosa, Pseudomonas stutzeri, Vibrio cholera, Vibrio vulnificus, Vibrio parahaemolyticus, Shigella flexneri, Yersinia enterocolitica, Yersinia pesti, Aeromonas salmonicida, Mycoplasma mycoides, Enterococcus faecalis, Yersinia pseudotuberculosis, Pasteurella multocida, Mycoplasma capricolum, Mycoplasma pneumonia, Mycoplasma hyorhinis, Mycoplasma mycoides, Mannheimia haemolytica, Salmonella enterica, E. coli KTE112, E. coli CFT073, E. coli K-12, Klebsiella pneumonia, Actinobacillus pleuropneumoniae, and Cronobacter sakazakii.

29. The cosmetic material according to claim 23, which includes mannitol at a concentration of 2.74 to 164 mM.
